(19) United States
(12) Patent Application Publication
(10) Pub. No.: US 2025/0072881 A1
(43) Pub. Date: Mar. 6, 2025

(54) DEVICES AND METHODS FOR OCCLUDING AN OPENING FORMED INTO A PATIENT

(71) Applicant: Boston Scientific Scimed, Inc., Maple Grove, MN (US)

(72) Inventors: Aiden Flanagan, Kilcolgan (IE); Bruce R. Forsyth, Hanover, MN (US)

(73) Assignee: Boston Scientific Scimed, Inc., Maple Grove, MN (US)

(21) Appl. No.: 18/817,557

(22) Filed: Aug. 28, 2024

Related U.S. Application Data
(60) Provisional application No. 63/579,083, filed on Aug. 28, 2023.

(51) Int. Cl. A61B 17/00; A61B 90/00; A61L 24/00; A61L 24/02; A61L 24/10

(52) U.S. Cl. CPC A61B 17/0057; A61B 90/39; A61L 24/001; A61L 24/02; A61L 24/104; A61B 2017/00623; A61B 2017/00628; A61B 2017/0065; A61B 2017/00818; A61B 2090/3933; A61B 2090/3966; A61L 2430/36

(57) ABSTRACT

Method and systems for closing an opening formed during a medical procedure are disclosed. An example method may include advancing a needle through patient tissue to form an opening into a bile duct region, performing an intervention within the bile duct region, and advancing an embolic gel into the opening to close the opening.

### CROSS-REFERENCE TO RELATED APPLICATIONS

This application claims the benefit of U.S. Provisional Patent Application Ser. No. 63/579,083 filed on Aug. 28, 2023, the disclosure of which is incorporated herein by reference.

### TECHNICAL FIELD

The present disclosure pertains to medical devices, and methods for using medical devices. More particularly, the present disclosure pertains to devices and methods for occluding an opening formed into a patient

### BACKGROUND

A wide variety of medical devices have been developed for medical use, for example, intravascular use. Some of these devices include guidewires, catheters, and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### BRIEF SUMMARY

This disclosure provides design, material, manufacturing method, and use alternatives for medical devices. A method for closing an opening formed during a medical procedure is disclosed. The method comprises: advancing a needle through patient tissue to form an opening into a bile duct region; performing an intervention within the bile duct region; and advancing an embolic gel into the opening to close the opening.

Alternatively or additionally to any of the embodiments above, performing an intervention within the bile duct region includes injecting a contrast agent into the bile duct region.

Alternatively or additionally to any of the embodiments above, performing an intervention within the bile duct region includes draining the bile duct region.

Alternatively or additionally to any of the embodiments above, performing an intervention within the bile duct region includes dilation of a biliary stricture.

Alternatively or additionally to any of the embodiments above, performing an intervention within the bile duct region includes delivery of a stent.

Alternatively or additionally to any of the embodiments above, performing an intervention within the bile duct region includes a tissue biopsy.

Alternatively or additionally to any of the embodiments above, further comprising disposing a stop member within the opening.

Alternatively or additionally to any of the embodiments above, disposing a stop member within the opening includes disposing the stop member adjacent to the bile duct region.

Alternatively or additionally to any of the embodiments above, disposing a stop member within the opening includes disposing the stop member within the opening prior to advancing the embolic gel into the opening.

Alternatively or additionally to any of the embodiments above, the stop member includes a stop pin having a head and a shaft extending from the head.

Alternatively or additionally to any of the embodiments above, the embolic gel includes a shear thinning material.

Alternatively or additionally to any of the embodiments above, the embolic gel includes a gelatin, a layered silicate mixture, and a radiopaque material.

A method for occluding an opening formed during a percutaneous transhepatic cholangiography procedure is disclosed. The method comprises: advancing a needle through patient tissue to form a drainage tract into a bile duct region; performing an intervention within the bile duct region; disposing a stop member within the drainage tract; and advancing a conformable embolic material into the drainage tract to occlude the drainage tract.

Alternatively or additionally to any of the embodiments above, the conformable embolic material includes a shear thinning material.

Alternatively or additionally to any of the embodiments above, the conformable embolic material includes a gelatin, a layered silicate mixture, and a radiopaque material.

Alternatively or additionally to any of the embodiments above, the stop member includes a stop pin having a head and a shaft extending from the head.

A system for occluding a drainage tract formed into a bile duct region as part of a percutaneous transhepatic cholangiography procedure is disclosed. The system comprises: a stop member configured to be disposed adjacent to the drainage tract formed into the bile duct region; a conformable embolic material configured to occlude the drainage tract; and a delivery device for delivering the conformable embolic material.

Alternatively or additionally to any of the embodiments above, the conformable embolic material includes a shear thinning material.

Alternatively or additionally to any of the embodiments above, the conformable embolic material includes a gelatin, a layered silicate mixture, and a radiopaque material.

Alternatively or additionally to any of the embodiments above, the stop member includes a stop pin having a head and a shaft extending from the head.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a schematic view of a bile duct region.

FIG. 2 illustrates a needle forming an opening to the bile duct region.

FIG. 3 is a schematic view of an opening formed into a bile duct region.

FIG. 4 illustrates a stop member disposed within an opening formed into a bile duct region.

FIG. 5 illustrates a stop member and an embolic gel disposed within an opening formed into a bile duct region.

FIG. 6 illustrates a stop member and an embolic gel disposed within an opening formed into a bile duct region.

FIG. 7 illustrates a stop member and an embolic gel disposed within an opening formed into a bile duct region.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (e.g., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the scope of the invention.

Percutaneous transhepatic cholangiography (PTC) is an interventional radiologic procedure that is usually performed when either endoscopic biliary access via endoscopic retrograde cholangiography is technically limited, the patient is not a good candidate for a typical biliary drainage procedure, or there is altered anatomy. PTC involves transhepatic insertion of a needle into a bile duct, followed by injection of contrast material to opacify the bile ducts. PTC localizes the site of obstruction and facilitates therapeutic interventions, such as biliary drainage, removal of bile duct stones, dilation of biliary strictures, stent placement, and tissue biopsies. A drainage procedure (e.g., percutaneous transhepatic biliary drainage) typically precedes the intervention. After a PTC has been performed a drainage catheter is re-inserted into the tract to avoid leakage of bile. After removing the drainage catheter, an opening into the biliary tract (e.g., the drainage tract) may still exist. It can be appreciated that closing and/or sealing the drainage tract may be desirable, for example in order to avoid leakage. Disclosed herein are systems and methods for closing and/or sealing a drainage tract, for example following a PTC procedure.

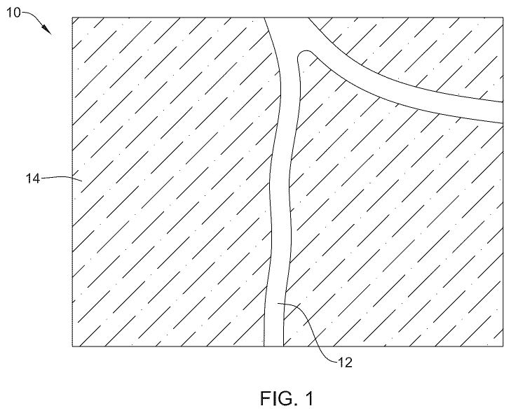

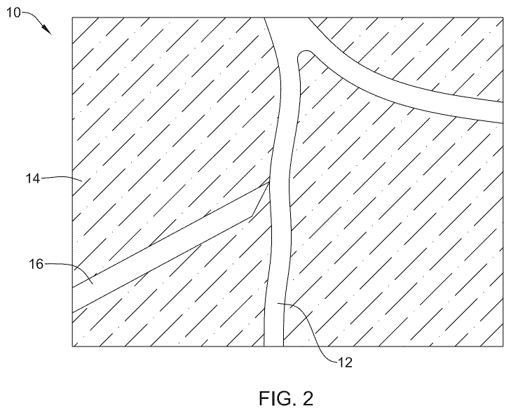

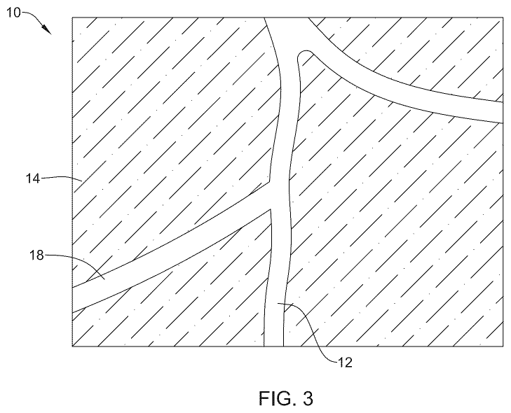

FIG. 1 schematically illustrates a patient target, generally noted with reference number 10. In this example, the target 10 includes a bile duct, a bile duct region, a biliary tract/tree, and/or the like. For the purposes of this disclosure, the term bile duct region, noted with reference number 12, will be used to denote such anatomical structures. It can be appreciated that the terms/names used to describe such anatomy and the graphic depiction thereof in the corresponding figures may be considered schematic in nature. During an intervention, a clinician may access the bile duct region 12 through patient tissue 14. For example, a PTC procedure may include the passing of a needle or catheter 16 through patient tissue 14 as schematically depicted in FIG. 2. The needle 16 can be removed. Removing the needle 16 may form an opening or drainage tract 18 to the bile duct region 12 as shown in FIG. 3.

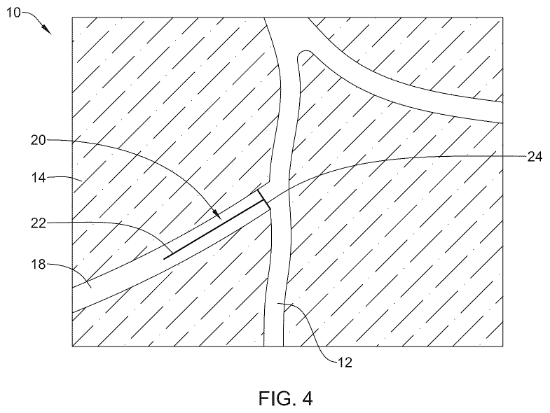

Following an intervention, it may be desirable to close and/or seal the drainage tract 18. A variety of procedures and devices are contemplated for achieving such closure/sealing. For examples, FIG. 4 illustrates a stop member 20 disposed in the opening. The stop member 20 may include a shaft 22 and a head 24. The stop member 20 may generally be configured for use with an embolic gel or the like. For example, the stop member 20 may form a backstop or end point that helps to define or limit migration of the embolic gel within the drainage tract 18 (e.g., so that the embolic gel closes the drainage tract 18 substantially without entering the bile duct region 12). The stop member 20 can vary in form. For example, the stop member 20 may take the form of or include a spiral or spiral region, a helix or helical region, a coil, a stent or stent-like structure, a stent or stent-like structure with a closed end, an expandable structure, an umbrella-like structure, combinations thereof, and/or the like. In some instances, the stop member 20 may be formed from a biodegradable or bioerodible material.

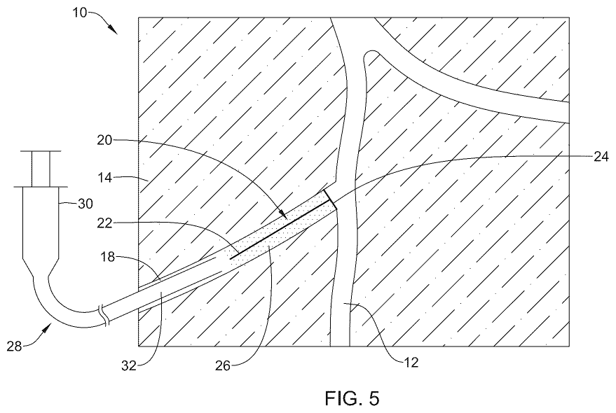

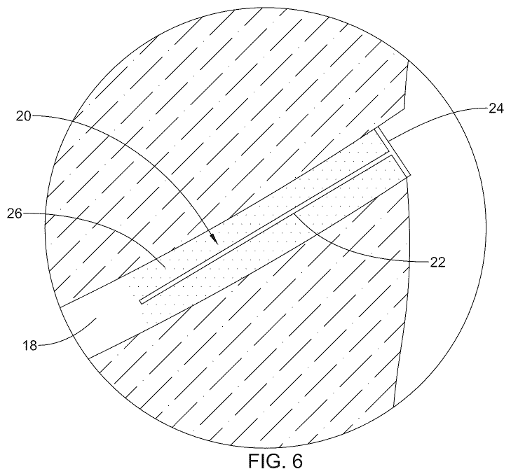

FIGS. 5-6 illustrate an embolic gel 26 disposed within the drainage tract 18 (e.g., adjacent to the stop member 20). In some instances, the embolic gel 26 may include a conformable embolic material. For example, the embolic gel 26 may comprise a shear thinning material or composition. The shear thinning material may include a gelatin or gelatin derivative and silicate nanoparticles. In some instances, the shear thinning material may also include a radiopaque material. For example, the embolic gel 26 may include OBSIDIO™ Conformable Embolic material, commercially available from Boston Scientific. In some of these and in other instances, the embolic gel 26 may include a hemostatic agent. For example, the embolic gel 26 may include chitosan (e.g., chitosan and a shear thinning material or composition such as OBSIDIO™ Conformable Embolic material).

The embolic gel 26 may be delivered to the drainage tract 18 using a suitable delivery device such as a catheter, syringe, a syringe coupled to a delivery tube or catheter, and/or the like. For example, FIG. 5 schematically illustrates a delivery device 28 including a syringe 30 and a tube or catheter 32. In instances where the embolic gel 26 is a shear thinning and/or conformable material, the embolic gel 26 may start as an injectable soft solid, flow as a liquid when force is applied, and returns to a soft solid to occlude the drainage tract 18. Thus, delivery of the embolic gel 26 may substantially occlude and/or seal the drainage tract 18, for example so that bile disposed within the bile duct region 12 is substantially prevented from entering the drainage tract 18 and/or leaking to other regions where the bile could lead to complications and/or damage. In some instances, the embolic gel 26 may be delivered to a region adjacent to the drainage tract 18 at a location adjacent to the bile duct region 12. In some of these and in other instances, the embolic gel 26 may be delivered along a portion or the full length of the drainage tract 18. For example, the embolic gel 26 may be delivered into the drainage tract 18 as the delivery device 28 is retracted from the drainage tract 18. This may result in the embolic gel 26 being disposed along the length of the drainage tract 18 (e.g., along the full length of the drainage tract 18).

The transition to a stable, occlusive form of the embolic gel 26 may occur in near real-time, meaning that sealing can be achieved quickly. After providing sufficient time for sufficient healing of the drainage tract 18, the embolic gel 26 will eventually hydrolyze within the patient. Therefore, no further procedures are necessary in order to remove objects implanted in the patient, nor are any long-term implants used. The use of the embolic gel 26 (e.g., where the embolic gel 26 is a shear thinning and/or conformable material) may be desirable for a number of reasons. For example, delivery of the embolic gel 26 to the drainage tract 18 may sufficiently close and/or seal the drainage tract 18 relatively quickly and stably. Thus, a patient treated with the embolic gel 26 may recover from a PTC procedure more quickly that patients that are treated with embolic coils and/or glues.

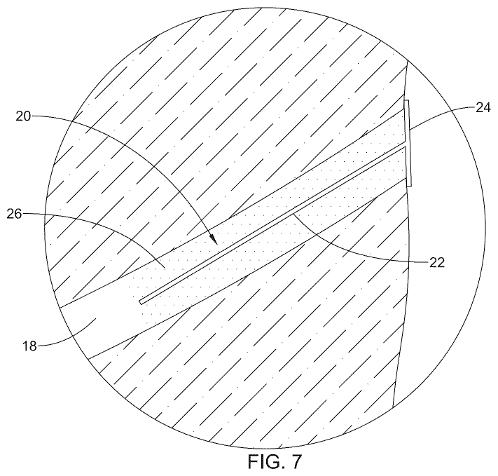

The positioning of the stop member 20 may vary, depending on the needs of the procedure and/or the preference of the clinician. For example, the stop member 20 may be arranged so that the head 24 is disposed at the junction of the drainage tract 18 and the bile duct region 12 (e.g., as shown in FIGS. 4-6). In other instances, the head 24 may be disposed so that it extends across the opening of the drainage tract 18 as shown in FIG. 7. Other arrangements are contemplated.

Some example methods are contemplated including methods for closing an opening formed during a medical procedure (e.g., methods for occluding an opening formed during a percutaneous transhepatic cholangiography procedure). The methods advancing a needle or catheter (e.g., the needle 16) through patient tissue to form an opening (e.g., the drainage tract 18) into a bile duct (e.g., the bile duct region 12). An intervention may be performed within the bile duct region. The intervention may include injecting a contrast agent into the bile duct region 12, draining the bile duct region 12, dilation of a biliary stricture (e.g., within the bile duct region 12), delivery of a stent (e.g., within the bile duct region 12), a tissue biopsy, combinations thereof, and/or the like. An embolic gel and/or conformable embolic material (e.g., the embolic gel 26) may be advanced into the opening (e.g., into the drainage tract 18) to close the opening. In some instances, a stop member (e.g., the stop member 20) may be disposed within the opening (e.g., the drainage tract 18), for example prior to advancing the embolic gel into the opening.

It can be appreciated that the devices and/or methods disclosed herein may also be applied to other interventions where an opening is formed in a patient. One example procedure may include a biopsy. Other procedures where the devices and/or methods disclosed herein may also be applied are contemplated.

U.S. Pat. No. 10,034,958 is herein incorporated by reference.

U.S. Pat. No. 11,083,780 is herein incorporated by reference.

U.S. Pat. No. 11,426,450 is herein incorporated by reference.

U.S. Patent Application No. US 2022/0192974 is herein incorporated by reference.

U.S. Patent Application No. US 2023/0093831 is herein incorporated by reference.

U.S. Patent Application No. US 2023/0094016 is herein incorporated by reference.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. A method for closing an opening formed during a medical procedure, the method comprising:
advancing a needle through patient tissue to form an opening into a bile duct region;
performing an intervention within the bile duct region; and
advancing an embolic gel into the opening to close the opening.

2. The method of claim 1, wherein performing an intervention within the bile duct region includes injecting a contrast agent into the bile duct region.

3. The method of claim 1, wherein performing an intervention within the bile duct region includes draining the bile duct region.

4. The method of claim 1, wherein performing an intervention within the bile duct region includes dilation of a biliary stricture.

5. The method of claim 1, wherein performing an intervention within the bile duct region includes delivery of a stent.

6. The method of claim 1, wherein performing an intervention within the bile duct region includes a tissue biopsy.

7. The method of claim 1, further comprising disposing a stop member within the opening.

8. The method of claim 7, wherein disposing a stop member within the opening includes disposing the stop member adjacent to the bile duct region.

9. The method of claim 7, wherein disposing a stop member within the opening includes disposing the stop member within the opening prior to advancing the embolic gel into the opening.

10. The method of claim 7, wherein the stop member includes a stop pin having a head and a shaft extending from the head.

11. The method of claim 1, wherein the embolic gel includes a shear thinning material.

12. The method of claim 1, wherein the embolic gel includes a gelatin, a layered silicate mixture, and a radiopaque material.

13. A method for occluding an opening formed during a percutaneous transhepatic cholangiography procedure, the method comprising:
advancing a needle through patient tissue to form a drainage tract into a bile duct region;
performing an intervention within the bile duct region;
disposing a stop member within the drainage tract; and
advancing a conformable embolic material into the drainage tract to occlude the drainage tract.

14. The method of claim 13, wherein the conformable embolic material includes a shear thinning material.

15. The method of claim 13, wherein the conformable embolic material includes a gelatin, a layered silicate mixture, and a radiopaque material.

16. The method of claim 13, wherein the stop member includes a stop pin having a head and a shaft extending from the head.

17. A system for occluding a drainage tract formed into a bile duct region as part of a percutaneous transhepatic cholangiography procedure, the system comprising:
a stop member configured to be disposed adjacent to the drainage tract formed into the bile duct region;
a conformable embolic material configured to occlude the drainage tract; and
a delivery device for delivering the conformable embolic material.

18. The system of claim 17, wherein the conformable embolic material includes a shear thinning material.

19. The system of claim 17, wherein the conformable embolic material includes a gelatin, a layered silicate mixture, and a radiopaque material.

20. The system of claim 17, wherein the stop member includes a stop pin having a head and a shaft extending from the head.
